(19) United States
(12) Patent Application Publication
(10) Pub. No.: US 2025/0306017 A1
(43) Pub. Date: Oct. 2, 2025

(54) LANTHANIDE ION CHELATE PARTICLES, KITS AND DIAGNOSTIC METHODS

(71) Applicant: IDEXX LABORATORIES, INC., Westbrook, ME (US)

(72) Inventors: Murthy Yerramilli, Westbrook, ME (US); Hongzhi Xie, Westbrook, ME (US); Asanterabi Malima, Westbrook, ME (US)

(21) Appl. No.: 19/092,211

(22) Filed: Mar. 27, 2025

Related U.S. Application Data
(60) Provisional application No. 63/571,890, filed on Mar. 29, 2024.

(51) Int. Cl. G01N 33/543

(52) U.S. Cl. CPC G01N 33/54346; G01N 2458/40

(57) ABSTRACT

Particles loaded with lanthanide (III) rare earth metal ions chelates for use as labels in binding assays. Particles and uses for detecting analytes in samples is described.

### RELATED APPLICATION

This application claims priority to U.S. Provisional Application No. 63/571,890 filed Mar. 29, 2024, which is incorporated herein by reference in its entirety.

### FIELD

The disclosure is directed to reagents, kits and methods for use in binding assays. More particularly, the reagents include lanthanide (III) rare earth metal ion (e.g. Europium) infused particles for use as fluorescent labels.

### BACKGROUND

The use of particles containing fluorescent dyes in binding assays is known.

However, such particles may not produce enough signal to be useful for advanced assay formats and systems that are intended to provide increased sensitivity. Accordingly, the present fluorescent particles provide increased signal and higher signal to noise ratios that allow the detection of analytes with a sensitivity in the one picomolar range.

### SUMMARY

One aspect of the present disclosure is directed to a particulate reagent including a first particle having a Europium 3+ (Eu3+) chelate complex loaded into the first particle, and a first binding partner for an analyte coated on the first particle, wherein (a) the first binding partner is directly coated on the first particle covalently or non-covalently, or (b) the first particle is directly coated covalently or non-covalently with streptavidin or neutravidin, and the first binding partner is bound to or becomes bound to the first particle through biotin/streptavidin or biotin/neutravidin interaction. In an aspect of the disclosure, the first particle that is directly coated covalently or non-covalently with streptavidin or neutravidin becomes bound to the first binding partner during an immunoassay. In another aspect, the particle includes a latex having a modified or unmodified surface. In another aspect, the first particle has a diameter of about 25 nm to about 150 nm. And in another aspect, the first binding partner is an antibody, an antibody binding fragment, or an aptamer. In yet another aspect, the particle is loaded with about 0.075 to about 0.125 mg of the Eu3+ chelate complex per mg of particle.

The disclosure is also directed to a kit including a particulate reagent of the disclosure and a second particle or a first surface including a second binding partner for an analyte. In various aspect of the disclosure, the second particle is about 100 to 1000 times the size of the first particle. In another aspect, the second particle has a cross-sectional surface area about 100 to 1000 times the size of a cross-sectional area of the first particle. In various aspects, the second particle comprises a bar-coded bead. Additional aspects include the first particle having a size between about 25 nm and 100 nm, and optionally the second particle having a size is between about 0.1 μm and 100 μm.

Another aspect of the disclosure is directed to a method of detecting the presence of a target analyte in a sample. The method includes forming a mixture of the sample with a second binding partner that is covalently or non-covalently bound to a second particle or to a first surface, contacting mixture with the particulate reagent of the various aspects of the disclosure, to form a labeled target analyte complex; and detecting the presence of the labeled target analyte complex by irradiating the complex with an excitation light source and detecting emitted light from the first particle. In various aspects, the excitation light source has a wavelength equal to or greater than 360 nm and detecting emission from the labeled target analyte complex. In addition, aspects include that the emission is detected using time-resolved fluorescence (TRF).

In various aspects of the disclosure, the Europium 3+ chelate complex includes a β-diketone compound with an ion of a lanthanide (III) rare earth metal as further described herein.

In another aspect of the disclosure, a complex comprises (1) an analyte, (2) a first particle comprising a lanthanide chelate complex, and further comprising a coating comprising a first binding partner for an analyte, wherein (a) the first binding partner is coated on the first particle covalently or non-covalently, or (b) the first particle is coated covalently or non-covalently with streptavidin or neutravidin, and the first binding partner is bound to or becomes bound to the first particle through biotin/streptavidin or biotin/neutravidin interaction, and (3) a second particle or a first surface comprising a second binding partner for an analyte. The second particle can be about 100 to 1000 times the size of the first particle. The second particle can have a cross-sectional surface area about 100 to 1000 times the size of a cross-sectional area of the first particle. The second particle can be a bar-coded bead. The first particle can be between about 25 nm and 100 nm in size, and, the second particle can be between about 0.1 μm and 100 μm in size.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure. No attempt is made to show structural details of the disclosure in more detail than may be necessary for a fundamental understanding of the disclosure and various ways in which it may be practiced.

FIG. 1 is a graph showing the results of an experiment to determine the stability of purified Eu3+ latex particles of the disclosure after several rounds of dialysis.

FIG. 2 is a graph showing a comparison in performance between a streptavidin-phycoerythrin conjugate (SA-PE) and the streptavidin coated Eu3+ particles of the disclosure (SA-Eu).

FIGS. 3A and 3B show graphs comparing the performance of the Eu3+ particles of the disclosure using time resolved fluorescence (TRF) (FIG. 3A) and standard fluorescence (FL) (FIG. 3B) measurements.

FIG. 4 shows the performance of serial percent solids dilutions for lots of Eu+3 particles when bound to barcoded magnetic beads.

FIG. 5 is a graph showing the results of an experiment to demonstrate the ability to measure variable concentrations of Eu3+ particles % solids on an electrowetting cartridge.

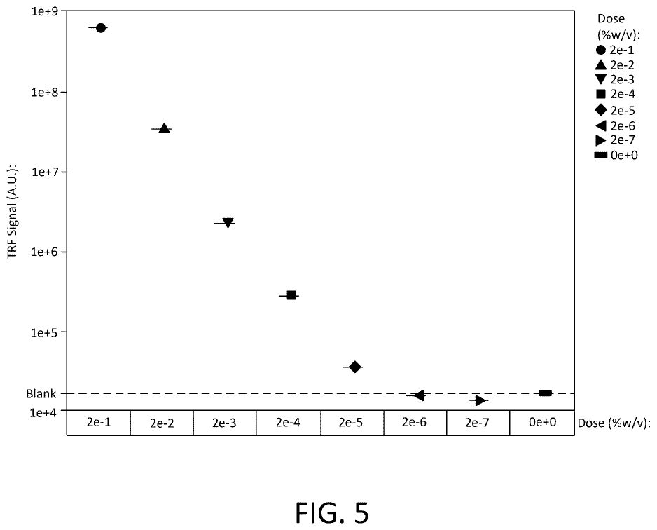

### DESCRIPTION

In various aspects, the disclosure is directed to particulate reagents including particles infused with a lanthanide ion chelate as a label that increases the detection signal and reduces the background noise for use in binding assays, such as immunoassays. In addition, aspects of the disclosure are directed to colloidal gold containing reagents that benefit from the binding partners as described herein to improve assay performance.

The particles forming the particulate reagent include polymer particles having an unmodified or functionalized surface chemistry that allows for covalent or non-covalent attachment of binding partners (e.g., antibodies, fragments thereof, proteins, aptamers, nucleic acids) to the particles. The particles should also allow for the loading of the lanthanide chelate as described herein. Example particles include carboxylate modified latex (CML) particles, for example polystyrene particles, functionalized to have a surface having a carboxy group that allows for conjugation of binding partners to the surface. Because of the way that the polystyrene chains arrange themselves in the particle, the surface is very hydrophobic in character, making these suitable materials for the attachment of materials such as proteins or other binding partners. In one aspect, these carboxylate modified latex (CML) particles are produced by copolymerizing carboxylic acid containing polymers that results is a latex polymer particle with a highly charged, relatively hydrophilic surface layer. In another aspect, particles that do not have a modified surface are not functionalized while allowing for passive or non-covalent coating of proteins and other reagents. For example, the surface allows non-covalent attachment of proteins, such as Bovine Serum Albumin (BSA) that can be used to conjugate various binding partners associated with an immunoassay.

In another aspect, the particulate reagent includes a colloidal gold particle having aggregates of gold associated with proteins, wherein a binding partner for the analyte is attached directly to the gold particle or indirectly as described herein (e.g., biotin-streptavidin/neutravidin interaction).

CML particles and non-functionalized latex particles are available from a number of manufacturers (e.g., Bangs Laboratories, Thermo Fischer) and in a variety of sizes. In embodiments, the size of the particles range from 25 nanometers (nm) to about 150 nanometers (nm), for examples, 25 nm, 50 nm, 75 nm, 100 nm, 125 nm, and 150 nm.

In embodiments, the particulate reagent of the disclosure includes particles that are infused with a lanthanide chelate that, in the embodiments of the disclosure, are exemplified as Europium 3+ as the lanthanide chelate. Such particles, referred to herein as “Eu3+ particles” or “Eu 3+ particles of the disclosure” are attached or become attached to binding partners for use in binding assays, for example immune assays involving binding between an analyte (e.g., ligand) and a binding partner or partners (e.g., antibody) specific for the analyte. Example embodiments of the particulate reagent includes streptavidin or neutravidin coated on the Eu3+ particles that provide for attachment of a biotinylated binding partner to the Eu3+ particle through biotin-streptavidin or biotin-neutravidin interaction. In other embodiments, a binding partner for the analyte (e.g. an antibody or binding fragment thereof) is directly attached to the particle, either covalently or non-covalently. Accordingly, the particulate reagent of the disclosure includes both particles having a binding partner for the analyte directly attached to the surface of the particle or a particle attached to neutravidin or streptavidin that allows for attachment of a biotinylated binding partner for the analyte. The attachment can occur once the particle and the binding partner are combined in an assay as described herein.

The particles of the disclosure provide improved signal response (dynamic range) in assays that rely on a fluorescent signal resulting in higher signal to noise rations due to background reduction. Such assays include, for example, Time-Resolved Fluorescence (TRF) assays and standard fluorescence assays. The Eu3+ particles, when used as labels for immunoassays according to the disclosure are capable of loading more Eu3+ chelate complexes compared to the particles described in U.S. Pat. No. 7,053,249, which, in some embodiments, results in increased signal and two to three orders magnitude improvement in TRF mode when compared to standard fluorescence mode measurements. For example, the particles of the disclosure can be loaded with about 1500 to 2500 Eu3+ chelate per single particle or about 0.075 to about 0.125 mg Eu3+-chelate complex per mg particle. The particles are also stable to harsh conditions resulting in increased stability in both liquid and dry phases. Harsh conditions, for example 60° C. and a high percentage or organic solvents, will denature enzymes or proteins.

The particulate reagent of the disclosure can be used in binding assays in any assay format that uses the detection of a fluorescent signal in the determination of the presence or amount of an analyte in a sample, for example biological, environmental or industrial samples. As used herein, the term “sample” may refer to either an unprocessed (“raw” or “native”) sample in any form (fresh, frozen, etc.), or “sample” may refer to a native sample that has been processed for analysis according to the disclosure (e.g., a processed sample), the difference between the samples apparent from the context of the disclosure. “Analytes” include substances in samples for which a binding partner can be prepared. Immunoassays using fluorescently-labeled binding partners (e.g., antibodies) are ubiquitous in the industry as well as methods and apparatuses that use nanoparticle labels for the determination of analytes in samples.

In one aspect, the particulate reagent of the disclosure provides a label for a first binding partner that binds to the analyte. In an embodiment, a second binding partner that also binds the analyte is immobilized on a solid phase or on a second particle for which association of the particles can be detected as a result of a fluorescent signal from the Eu3+ particle. The second particle may be in solution or is or can be immobilized on a solid phase. In an example embodiment, the second particle is a magnetic particle such as a barcoded magnetic bead wherein the barcode identifies the second binding partner on the bead.

In one aspect, barcoded magnetic beads are used in conjunction with the particulate reagent of the disclosure in order to identify one or more analytes in a sample. Because the barcode on the magnetic bead can identify the binding partner associated with the barcoded bead, embodiments of the disclosure include one or more analyte binding partners bound to identical particles of the disclosure, which may be used in multiplex assay format. In embodiments, multiple different analytes, once labeled with the same label, may be identified as a result of the binding of the analyte to a barcoded magnetic bead that provides an identification of the binding partner on the bead that can be recognized by the optical decoder.

Examples of barcoded magnetic beads and associated systems may be found in U.S. Pat. Nos. 7,745,091, 8,148,139, and 8,614,852, and U.S. Pub. Nos. US 2023/0184636 and US 2023/0181421, each of which is incorporated by reference herein in its entirety.

Barcoded magnetic beads can be decoded by systems employing an optical scanner decoder that can recognize the barcodes. Such systems include, for example, a BIOCODE® 2500 Analyzer (Applied BioCode Inc.) This analyzer is a multiplex immunoassay reader that detects the barcodes and the fluorescent signal of the beads on the bottom of reaction wells. Another example system includes a multiplex analyzer which is capable of decoding barcoded magnetic beads along with detection fluorescent signal (e.g., BIOCODE® 2500 analyzer). Another example system includes the multiplex diagnostic platform that relies on electrowetting to process and read barcoded magnetic beads is described in U.S. Patent Application Nos. 63/482,240 and 63/484,433, each of which are incorporated by reference herein in its entirety.

In various aspects, the disclosure is directed to a particulate reagent that includes a first particle loaded with a lanthanide chelate complex, for example a Europium 3+ chelate complex. In one aspect and without wishing to be bound by theory, particle loading is believed to be the result of softening a latex particle in the presence of an organic solvent and the chelate complex, which is believed to allow the chelate to penetrate the latex and migrate to a hollow center of the particle. Once the particles are loaded, the solvent can be evaporated and the particles resuspended by diluting the particles in aqueous salt solution that re-hardens the latex and traps the chelate in the particle. The dilute solution can be dialyzed one or more times to remove excess chelate. In one aspect, the disclosure is directed to the use of a highly dilute solution of the particles in the salt solution to increase the loading of the lanthanide chelate into the particle.

A first binding partner for the analyte (e.g., an anti-analyte antibody for an antigen or binding fragment thereof) is associated with the first particle either directly or through binding between biotin and a biotin-binding protein, such as, for example streptavidin and neutravidin. For example, in one embodiment, the first binding partner is directly coated on the first particle covalently or non-covalently. In another example embodiment, the first binding partner is biotinylated, and the first particle has streptavidin or neutravidin covalently attached thereto. The first binding partner is bound to or becomes bound to the first particle through biotin/streptavidin or biotin/neutravidin interaction. The interaction may occur in the presence or absence of the analyte. For example, the interaction may occur prior to the particulate reagent being added to a reaction mixture including the sample or the sample being added to a reaction mixture including the particulate reagent. In other embodiments, the sample, the first biotinylated binding partner and the streptavidin or neutravidin coated particle can be combined sequentially or simultaneously in any order. The Eu3+ particle of the disclosure becomes bound to the second particle as a result of the formation of a complex (e.g., an immune complex) through binding between the first binding partner for an analyte, the analyte, and a second binding partner for the analyte that is attached to a second particle.

In embodiments of the disclosure, the Eu3+ particle (first particle) has a smaller in size (e.g., measured by diameter or surface area) than a particle attached to a second binding partner, for example a barcoded magnetic bead. In example embodiments, the first particle is between about 25 nm and 400 nm in size, for example about 25 to 50 nm, about 25 to 75 nm, or about 25 to 100 nm. Barcoded magnetic beads (BMBs) can be any shape but generally include at substantially planar surface that includes a barcode that can be read by an appropriate reader. For example, BMBs may be cylindrical wherein the diameter of the cylinder is significantly more than the high of the cylinder, rendering the BMB having a disc-like shape. In another embodiment, the BMB is rectangular having X and Y dimensions substantially more than a Z dimension, which provides a cuboid-type particle. Dimensions of such particles can be, for example, about 0.1 μm and 100 μm. As an example, a BMB may have X-Y-Z dimensions of about 70, 40, 5 microns (having an approximate surface area of 6.7 mm squared) is about two to three orders of magnitude larger (100 to 1000 times) larger than 50 nm particles according to the disclosure (having an approximate surface area of 8 microns). If only the largest plane of a BMB having 70, 40, 5 micron dimensions (e.g., about 2800 microns squared) is measured and compared to a the area of the central diameter plane of an example 25 nm particle (about 490 nm squared), the total area of the plane of the BMB is about two to three orders of magnitude larger (about 100 to 1000 times larger) than the central plane the particle. In this situation, hundreds or thousands of Eu3+ particles of the disclosure can be bound to the surface of a BMB of other particle having a similar difference in size from the Eu3+ particles.

In another aspect, the disclosure is directed to a kit including the Eu3+ particulate reagent of the disclosure. Kits include the particulate reagent and other reagents for conducting an immunoassay, including a binding partner for the analyte, which may be directly bound to the particle or, in some aspects of the disclosure, a streptavidin- or neutravidin-coated Eu3+ particle is packaged in a separate container from a biotinylated binding partner for the analyte. The kit may also include a second particle or a first surface, either including a second binding partner for an analyte as described herein. In some embodiments, the first surface may be associated with a multi-well plate or an analytic cartridge as described herein.

In another aspect, the disclosure is directed to a method of detecting the presence of a target analyte in a sample. The method includes reacting the sample with a second binding partner that is associated with a second particle (e.g., barcoded magnetic bead) or a first surface, and then reacting the sample with the particulate reagent as described herein. In another embodiment, the order of combination can be performed in any order. For example, a first particle that has been functionalized with streptavidin or neutravidin is contacted with a biotinylated binding partner for the analyte, either before, after, or simultaneously with contacting a sample with the biotinylated binding partner. An immune complex including the first particle, biotinylated binding partner, and the analyte is then contacted with the second particle or first surface that includes the second binding partner for the analyte. The presence of the first particle can then be determined by irradiating the complex with an excitation light source and detecting emitted light from the first particle.

In embodiments of the disclosure, the detection of the first particle includes analysis of the fluorescent signal from the first binding partner either directly or through time-resolved fluorescence (TRF). In embodiments, the excitation light source has a wavelength equal to or greater than 360 nm.

Lanthanide chelates, such as Europium chelates, for use as fluorescent labels are disclosed, for example, in U.S. Pat. No. 7,053,249, which provides examples of a number of embodiments of such chelates including a Europium 3+ (Eu3+ or Eu3+) chelate complex including a β-diketone compound having one of the following formulas:

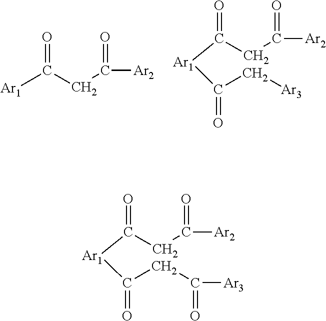

in which Ar₁ is an aromatic, heterocyclic aromatic or a substituted heterocyclic aromatic group; and Ar₂ and Ar₃ are independently selected from monocyclic aryl groups and multi-cyclic aryl groups.

In example embodiments, Ar₁ is an aromatic, heterocyclic aromatic or a substituted heterocyclic aromatic group, substituted with C₁-C₅ alkyl, C₁-C₅ alkenyl, C₁-C₅ alkynyl groups, or with substituted C₁-C₅ alkyl, C₁-C₅ alkenyl, C₁-C₈ alkynyl groups; and Ar₂ and Ar₃ are independently selected from monocyclic aryl groups and multi-cyclic aryl groups. In example embodiments, the β-diketone compound Ar₁ is a naphthyl, anthracenyl, or phenanthrenyl group or a substituted naphthyl, anthracenyl, or phenanthrenyl group, and/or the β-diketone compound Ar₂ is a substituted phenyl group, for example, the Ar₂ is a phenyl group substituted by one or more of: —F; —CF₃; —N(CH₃)₂.

In example embodiments of the β-diketone compound either Ar₁ or Ar₂ or both are substituted 2′ to the diketone substituent with a moiety having the formula:

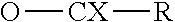

wherein X is S or O, and R is O—R₁ or S—R₁, where R₁ is C₁-C₅ alkyl, C₁-C₅ alkenyl, C₁-C₅ alkynyl groups, or substituted C₁-C₅ alkyl, C₁-C₅ alkenyl, C₁-C₅ alkynyl groups. For example, in the β-diketone compound
Ar₁ is an aromatic, heterocyclic aromatic or a substituted heterocyclic aromatic group; and
Ar₂ and Ar₃ are independently selected from monocyclic aryl groups and multi-cyclic aryl groups and wherein either Ar₁ or Ar₂ or both are substituted 2′ to the diketone substituent with a moiety having the formula:

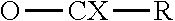

wherein X is S or O, and R is O—R₁ or S—R₁, where R₁ is C₁-C₈ alkyl, C₁-C₅ alkenyl, C₁-C₅ alkynyl groups, or substituted C₁-C₈ alkyl, C₁-C₅ alkenyl, C₁-C₈ alkynyl groups.

In other example embodiment, the β-diketone compound Ar₁ is a 2′ naphthyl group or may include 1-(3,5-difluorophenyl)-3-(naphth-2-yl) propane-1,3-dione.

In any one of the foregoing embodiments, the chelate complex can be loaded into the first particle and can further include a non-fluorescent chelator, for example, tri-n-octyl-phosphene oxide (TOPO).

### EXAMPLES

The examples that follow are illustrative of specific embodiments of the disclosure. They are set forth for explanatory purposes only and are not intended to limit the scope of the disclosure.

### Example 1

### Preparation of Eu CML Latex Particles:

1) 18.6 mg of Difluoro-Phenyl-naphthyl-propanedione (DF-PNPD) (MW 310, 0.03 mmol) dissolved in 30 ml of dioxane (1 mM), was mixed with 34.8 mg of Trioctylphosphine oxide (TOPO) in 90 ml of methanol (1 mM), and 30 ml of 1 mM Eu(III) chloride mixed with 190 ml of methanol.
2) After 5 mins, this mixture was diluted with 190 ml of deionized water and shaken for 30 min to allow complete formation of the chelate.
3) Simultaneously, 4 ml of 50 nm CML latex particles (Bangs 10% solids, Catalog No. PC02220, Lot #C981838) was added to 12 ml deionized water, mixed with 40 μl of 6 N NaOH solution and 16 ml of methanol, and incubated for 30 mins.
4) The chelate solution (from step 2) and the CML latex particles (from step 3) were mixed together along with 80 μl of 6 N NaOH and shaken overnight at room temperature.
5) The mixture was concentrated to about 75 ml on an evaporator on at 45° C. under vacuum.
6) The concentrated particle suspension was dialyzed against 5 L of 10 mM sodium chloride in deionized water five times for about 6 hours for each dialysis, changing the dialysis buffer twice a day.
7) The final Eu3+ coated CML particles were obtained in 10 mM NaCl in water.

The foregoing procedure enhances the loading of Eu-chelate into latex particles and accommodates more Eu-particles on barcoded magnetic beads. Without wishing to be bound by theory, it is believed that one factor contributing to this result is that smaller carboxyl latex particles (CML) with a size of 50 nm are used. As noted above, after encapsulating the particles with Eu-chelate in a mixed organic and water solvent, the organic solvents were completely removed. Subsequently, particle concentration was increased by removing ⅔ of the originally added water. This method allows the loading of 2000 units of Eu-chelate into single particles at a concentration of 0.34% (w/v) and a Eu3+ concentration of 159 μM. This is in contrast the procedure described in U.S. Pat. No. 7,053,249B2, which describes 104 nm size of particles and one-half of the originally added water removed during the coating process.

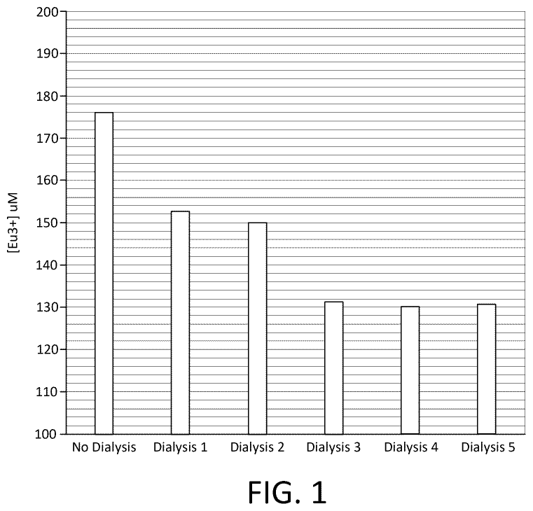

FIG. 1 shows that free Eu3+ was effectively removed through two cycles of dialysis against 10 mM NaCl, maintaining consistency after three cycles. This reflects that the Eu3+ ions were successfully encapsulated within the latex particles. Even after an additional two rounds of dialysis in 10 mM NaCl, each lasting 1 day at room temperature, the encapsulated Eu3+ remained securely within the latex particles. The results suggest that the purified Eu-latex particles exhibit stability, with no observable leakage of Eu3+ into the solution following encapsulation.

### Example 2: Preparation of Biotin-Linked Neutravidin Coated Eu-Particles

10% BSA solution (3 gr/30 ml of DIW) was filtered through a 0.2 μm filter and dialyzed against 4 L of deionized water. One ml of the dialyzed BSA (63.5 mg/ml) was diluted with 7 ml of 50 mM sodium phosphate buffer pH 8.0 and mixed with 0.5 ml of NHS-X-biotin (Pierce Chemical Company) solution (20 mg/ml) in anhydrous DMF. The reaction was allowed to proceed for 3 hours while stirring. After 3 hours, the reaction mixture was dialyzed against 6 L of 20 mM TRIS pH 7.4 to get rid of the unreacted biotin.

Coating of Eu-CML Particles with Biotin-BSA:

3 ml of Eu-CML particles prepared as in Example 1 (−0.4% solids) in 10 mM NaCl in deionized water were mixed with 60 μl of 1 M TRIS pH 7.4 and 2.5 ml of biotin/BSA (7.15 mg/ml in 20 mM TRIS pH 7.4) in a polypropylene tube and put on rotator at 4° C. overnight. Next morning, the particles were spun at 15 K rpm for 30 minutes. The pellet was resuspended in 2 ml of 20 mM TRIS pH 7.4 and spun again. This process was repeated twice. Finally, the particles were suspended in 2 ml of 20 mM TRIS pH 7.4.

Coating of Biotin-BSA-Coated Particles with Neutravidin:

2 ml of above biotin-BSA coated particles in 20 mM TRIS pH 7.4 were mixed with 0.4 ml of 10 mg/ml solution of neutravidin in the same buffer and put on rotator at 4° C. for 48 hours. After 48 hours, the unbound protein was removed by centrifuge and washing, and the particles were finally suspended in 20 mM TRIS pH 7.4.

### Example 3: Direct Coating Neutravidin/Streptavidin on Eu-CML Particles

12 ml of Eu-CML particles prepared as in Example 1 (0.34%) was adjusted to final pH as 20 mM at pH 7.4 by using 1 M Tris buffer (pH 7.4). Then the particle suspension was added with slow shaking into 1.0 ml streptavidin or neutravidin solution in Tris (5 mg/ml in 20 mM at pH 7.4) and the mixture was rotated overnight at 4° C. The particle suspension was separated into 1.5 ml vials and centrifuged at 14000 rpm for 1.5 hours at 4° C. The neutravidin/streptavidin residue in supernatant was measured by protein concentration measurement kit to determine the coating efficiency. The particles were blocked with BSA by resuspending the particles into 3.5 ml of Tris buffer (20 mM at pH 7.4) with 1% BSA for 1 hour at 25° C. and then overnight at 4° C. The final neutravidin or streptavidin coated and BSA blocked particles were centrifuged again at 14000 rpm for 1 hour to remove the supernatant, and the particle pellet was washed twice with tris buffer (20 mM at pH7.40) and resuspended in the same buffer.

### Example 3: Direct Coating of Antibody on Eu-CML Particles

5 ml of Eu-CML particles prepared as in Example 1 were pH adjusted by adding 0.25 ml bicarbonate buffer (1M at pH 9.0). To each the Eu-CML particle suspension, 0.36, 0.73 or 1.4 ml of anti-hookworm antibody (recombinant RDX17 antibody, part number 14-0035348-00, IDEXX Laboratories, Inc.) solution in bicarbonate buffer (3.6 mg/ml in 50 mM at pH 9.0) was added respectively. The suspension was then rotated overnight at 4° C. 0.5 ml of the particles was centrifuged at RPM=14000 for 1.0 hour at 4° C. to determine the antibody coating efficiency. The particles suspension was added BSA at final concentration of 0.5% and blocked at 4° C. overnight. The suspension was centrifuged at 14,000 RPM for one hour and then the supernatant was removed, and the pellet was washed with Tris buffer (20 mM at pH 7.40 with 0.05% Tween 20) three times. The final pellet was resuspended in Tris buffer (20 mM at pH 7.4 with 0.05% Tween 20) and ready for use.

### Example 4: Ab-Eu3+ Particles as Conjugates on ELISA Plate Assay when Compared SA-PE

This experiment demonstrated the use of RDX-17-Eu3+ particles prepared as in Example 3 compared to phycoerythrin (PE) dye for detecting hookworm antigen.

Procedure: Streptavidin conjugated to PE was obtained from Moss, Inc. (Catalog No. SAP3-001) (Pasadena, Maryland, USA).

Sample antigen was made by spiking recombinant antigen spiked in Giardia buffer (IDEXX part number is 45-0013750-00).

Assay reagents were placed at room temperature (RT) for 1 hour prior to dilution in MPx assay buffer (IDEXX Laboratories, part No. 25-0035312-00). Then, 100 μL of diluted sample concentrations (0, 10 and 1000) was added to wells of commercially available hookworm ELISA plates (IDEXX Laboratories, Part No. 49-0001885-00) followed by adding 50 μL of RDX-17-Eu3+ or the PE-hookworm antibody conjugate as described above.

The plate was shaken a 1000 RPM for 1 hour. After the incubation, the plate was washed 20 times using the standard MPx PBS-T buffer (phosphate buffer with 0.05% Tween 20) (IDEXX Part No. 43-0013471-00). The plate was then mounted on a SpectraMax i3 reader (Molecular Devices) for TRF measurements (excitation: 370 nm/emission: 616 nm) for the SA-Eu particles and for standard fluorescence measurement for the SA-PE reagent (excitation 520 nm/emission 590 nm).

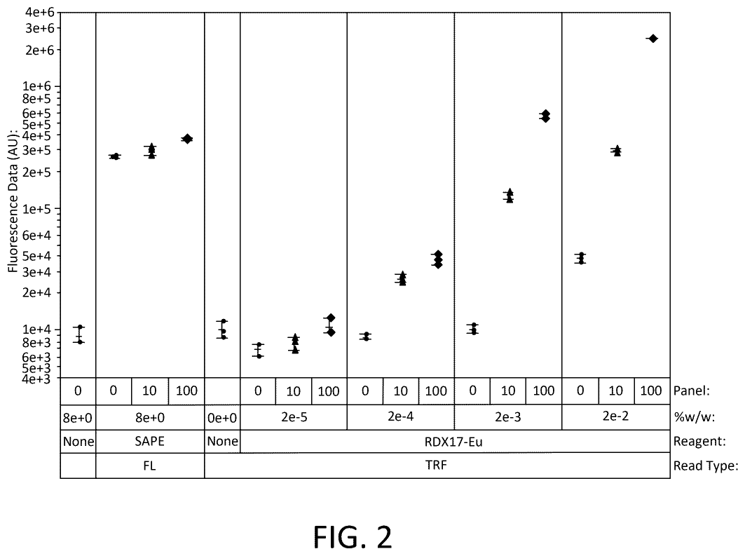

The results indicate that RDx-17-Eu3+ particles prepared as in Example 3 exhibit superior performance compared to PE-conjugate. This is manifested through the enhancement of signal intensity and significant reduction of the background noise. FIG. 2 shows a comparison of the performance comparisons between PE and Eu when used as conjugates on plates.

In addition, Eu3+ measurements were compared between standard fluorescence and TRF for the Eu3+ particles described above. TRF measurements and standard fluorescence were determined on the Spectramax reader.

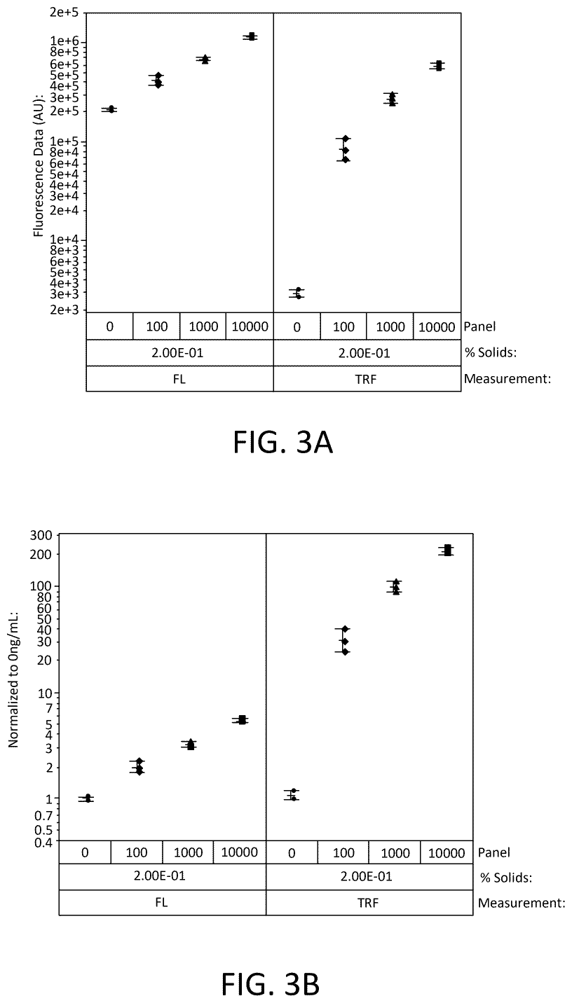

FIG. 3A shows the TRF and standard fluorescence reading obtained on the reader. FIG. 3B reflects that data normalized to 0 ng/ml shows approximately 50 times improvement in dynamic range (right panel).

### Example 5: Feasibility of Eu3+ Particles on a Multiplex (MPx) Platform with Barcoded Magnetic Beads

This experiment demonstrates the binding of biotinylated-Eu3+ particles (prepared as in Example 1) on streptavidin-coated barcoded magnetic beads (SA-BMBs) (IDEXX Part no. 21-0035507-00). Three different lots of Eu3+ particles serially manufactured approximately two months apart were used to determine stability.

Reagents were placed at room temperature (RT) for 1 hour. BMB solutions were then diluted at specified dilutions (1e-1% (0.1%) to 1e-3% (0.001%) solids) in separate tubes and placed onto tube shaker for about 2 minutes at 1000 RPM. While tubes were shaking, an Integra pipettor was used to dispense 100 uL into each designated well on a standard UV plate. 50 uL of aspirated volume was removed from each well by placing the UV plate on magnetic strip plate holder. 50 uL of BSA-BT-Eu particles were added to the wells and placed on a plate shaker at 1000 RPM for ten minutes. The plate was then washed the plate with MPX protocol 20 times using PBS-T buffer. Time Resolved Fluorescence (TRF) reads were taken with the SpectraMax i3 reader.

In order to assess the extent to which the BMB contribute to the signal, 200 uL of assay read buffer (IDEXX Laboratories, part No. 42-0035438-00) was added to a standard UV microplate and bead counts were taken using a Biocode 2500 reader. The plate was washed once with the MPx PBS-T buffer and the contents of each well were transferred into a new, non-binding black 96-well plate. TRF reads were taken using the SpectraMax i3 reader. The beads were transferred back to the original plate in the presence of 200 uL of read buffer (IDEXX Laboratories, part No. 42-0035438-00) and bead counts were obtained using the Biocode® 2500 analyzer to normalize the fluorescence per bead signal.

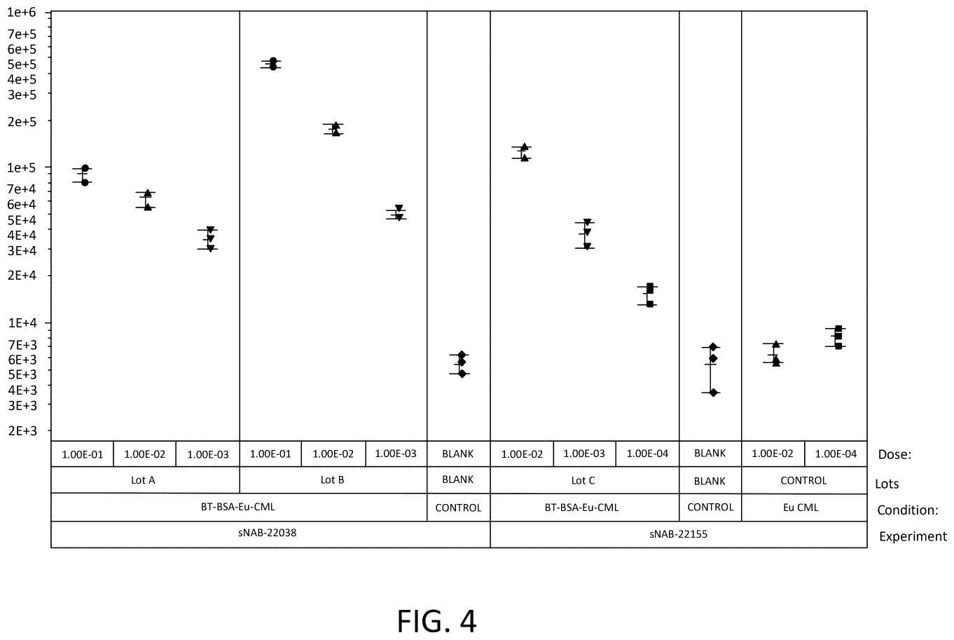

This work was repeated using three different lots serially manufactured about two months apart and compared to control (non-biotinylated Eu3+ lot) as shown in FIG. 4, which demonstrates the binding potential and signal dosing of Eu3+ particles on BMBs on the MPx platform provided sufficient dose response and signal to noise ratios between the three lots.

### Example 6: Measurement of Variable Concentrations (solids) of Eu3+ Particles in a Droplet on the Electrowetting Cartridge

This experiment tested the use of Eu³⁺ reagents on a cartridge that relies on electrowetting on a dielectric (EWOD) for sample processing and analysis as described in U.S. Patent Application Nos. 63/482,240 and 63/484,433.

A stock solution of 1.04% w/v of Neutravidin-Eu3+ particles prepared as in Example 1 was serially diluted from 2e-1% (0.2%) down to 2e-7% (0.0000002%) w/v percent solids using PBS-T Buffer (0.05% Tween-20 was added for electro-wetting purposes). 6 μL of Neutravidin-Eu3+ particle dilutions were loaded into each s-lane reservoir (2e-1% to 2e-7% left to right) of a FINDER® Chemistry Development Cartridge (Ref: 4186, Baebies, Inc.) and the cartridge was loaded onto a Baebies FINDER® 1.5 instrument. The EWOD was initiated using a setup script to check the cartridge health (impedance check) and to turn on voltage for loading reagents into s-lanes (vertical lanes). Electrowetting was started by dispensing two droplet units (DU, approximate 1 μL each) of the dilutions, and the droplets were electro-wetted to the center of cartridge.

The power to the FINDER instrument was turned off (run aborted), and the cartridge was carefully brought to the SpectraMax i3. TRF measurements of diluted reagents were taken on the cartridge. Dosing was observed with fluorescence intensity ranging from 8×10⁸ (800 million) arbitrary units (AU) down to 20,000 AU. This example demonstrated the ability to measure variable concentrations of Eu3+ particles % solids in a 2DU droplet on the EWOD cartridge.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A particulate reagent comprising:
a first particle having a lanthanide chelate complex loaded into the first particle, and
a first binding partner for an analyte coated on the first particle, wherein
(a) the first binding partner is directly coated on the first particle covalently or non-covalently, or
(b) the first particle is directly coated covalently or non-covalently with streptavidin or neutravidin, and the first binding partner is bound to or becomes bound to the first particle through biotin/streptavidin or biotin/neutravidin interaction.

2. The particulate reagent of claim 1 wherein the first particle is directly coated covalently or non-covalently with streptavidin or neutravidin becomes bound to the first binding partner during an immunoassay.

3. The particulate reagent of claim 1, wherein the first particle comprises a latex having a modified or unmodified surface.

4. The particulate reagent of claim 1, wherein the first particle has a diameter of about 25 nm to about 150 nm.

5. The particulate reagent of claim 1, wherein the first binding partner is an antibody, an antibody binding fragment, or an aptamer.

6. The particulate reagent of claim 1, wherein the lanthanide chelate is a Europium 3+ chelate.

7. The particulate reagent of claim 6, wherein the particle is loaded with about 0.075 to about 0.125 mg of the Eu3+ chelate complex per milligram of particle.

8. A kit comprising the particulate reagent of claim 6 and a second particle or a first surface wherein the second particle or first surface comprises a second binding partner for an analyte.

9. The kit of claim 8, wherein the second particle is about 100 to 1000 times the size of the first particle.

10. The kit of claim 9, wherein the second particle has a cross-sectional surface area about 100 to 1000 times the size of a cross-sectional area of the first particle.

11. The kit of claim 9, wherein the second particle comprises a bar-coded bead.

12. The kit of claim 8, wherein the first particle is between about 25 nm and 100 nm in size, and the second particle is between about 0.1 μm and 100 μm in size.

13. The particulate reagent of claim 1, wherein, the lanthanide chelate complex comprises a β-diketone compound with an ion of a lanthanide (III) rare earth metal wherein said β-diketone compound has one of the following formulas: 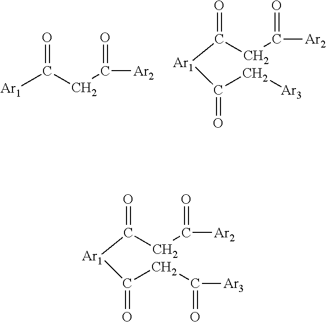
in which Ar1 is an aromatic, heterocyclic aromatic or a substituted heterocyclic aromatic group; and Ar2 and Ar3 are independently selected from monocyclic aryl groups and multi-cyclic aryl groups.

14. The particulate reagent of claim 13, wherein
Ar₁ is an aromatic, heterocyclic aromatic or a substituted heterocyclic aromatic group, substituted with C₁-C₅ alkyl, C₁-C₅ alkenyl, C₁-C₅ alkynyl groups, or with substituted C₁-C₅alkyl, C₁-C₅ alkenyl, C₁-C₅alkynyl groups; and
Ar₂ and Ar₃ are independently selected from monocyclic aryl groups and multi-cyclic aryl groups.

15. The particulate reagent of claim 13, wherein for the β-diketone compound Ar₁ is a naphthyl, anthracenyl, or phenanthrenyl group or a substituted naphthyl, anthracenyl, or phenanthrenyl group.

16. The particulate reagent of claim 13, wherein for the β-diketone compound Ar₂ is a substituted phenyl group.

17. The particulate reagent of claim 16, wherein the Ar₂ is a phenyl group substituted by one or more of: —F; —CF₃; —N(CH₃)₂.

18. The particulate reagent of claim 13, wherein for the β-diketone compound either Ar₁ or Ar₂ or both are substituted 2′ to the diketone substituent with a moiety having the formula: 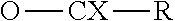
wherein X is S or O, and R is O—R₁ or S—R₁, where R₁ is C₁-C₅ alkyl, C₁-C₅ alkenyl, C₁-C₅alkynyl groups, or substituted C₁-C₅ alkyl, C₁-C₅ alkenyl, C₁-C₅ alkynyl groups.

19. The particulate reagent of claim 13, wherein for the β-diketone compound
Ar₁ is an aromatic, heterocyclic aromatic or a substituted heterocyclic aromatic group; and
Ar₂ and Ar₃ are independently selected from monocyclic aryl groups and multi-cyclic aryl groups and wherein either Ar₁ or Ar₂ or both are substituted 2′ to the diketone substituent with a moiety having the formula: 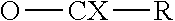
wherein X is S or O, and R is O—R₁ or S—R₁, where R₁ is C₁-C₅ alkyl, C₁-C₅ alkenyl, C₁-C₅ alkynyl groups, or substituted C₁-C₅ alkyl, C₁-C₅ alkenyl, C₁-C₅ alkynyl groups.

20. The particulate reagent of claim 13, wherein for the β-diketone compound Ar₁ is a 2′ naphthyl group.

21. The particulate reagent of claim 13, wherein the β-diketone compound comprises 1-(3,5-difluorophenyl)-3-(naphth-2-yl) propane-1,3-dione.

22. The particulate reagent of claim 1, wherein the complex loaded into the first particle further comprises a non-fluorescent chelator.
